(19) United States
(12) Patent Application Publication
(10) Pub. No.: US 2021/0375416 A1
(43) Pub. Date: Dec. 2, 2021

(54) Method of Distributing FDA Approved Prescribing Information

(71) Applicant: Chartwell Pharmaceuticals LLC, Congers, NY (US)

(72) Inventor: Jack Goldenberg, Congers, NY (US)

(21) Appl. No.: 17/226,298

(22) Filed: Apr. 9, 2021

Related U.S. Application Data
(60) Provisional application No. 63/032,890, filed on Jun. 1, 2020.

(51) Int. Cl. G16H 20/10; A61J 1/03; B65D 77/04; B65D 77/28; G06K 19/06

(52) U.S. Cl. CPC G16H 20/10; A61J 1/03; B65D 77/04; B65D 2577/043; G06K 19/06037; A61J 2205/10; B65D 77/28

(57) ABSTRACT

A method of providing a container containing at least one prescription medication package and its approved Food and Drug Administration prescribing information is described. The Food and Drug Administration prescribing information is not affixed to, adhered to, attached to, or contained within the package of medication, and is present in a quantity that is equal to, or greater, than the quantity of prescription medication packages.

### CROSS-REFERENCE TO RELATED APPLICATIONS

This application claims the benefit of U.S. Provisional Application No. 63/032,890 filed Jun. 1, 2020, which is hereby incorporated herein by reference in its entirety.

### TECHNICAL FIELD

The present invention relates to a method for distributing prescription medication packages along with their approved Food and Drug Administration prescribing information.

### BACKGROUND ART

In the United States, the Food and Drug Administration (“FDA”) requires that FDA approved prescribing information be included with each prescription medication package that is distributed by the manufacturer of the medication. The FDA approved prescribing information provides professional information (indications and usage, contraindications, warnings, and drug interactions, for example) about a medication to medical professionals, such as doctors and pharmacists.

Presently, medication manufacturers fold a sheet of paper containing the FDA approved prescribing information for a given prescription medication and affix the folded sheet of paper to an individual package of the given prescription medication, e.g., by gluing the folded sheet of paper to the individual package.

The process of folding each sheet of the FDA approved prescribing information and affixing each folded sheet to a prescription medication package is costly and time consuming.

### SUMMARY OF THE EMBODIMENTS

In accordance with one embodiment of the invention, a method of providing a quantity of packages of prescription medication and approved Food and Drug Administration prescribing information specific for the packages of prescription medication, the method comprises the steps of providing, in a container, the quantity of packages of prescription medication and providing, in the container, a quantity of members, each member comprising the approved Food and Drug Administration prescribing information specific for the packages of prescription medication, wherein the quantity of the members is equal to, or greater than, the quantity of packages of prescription medication, and wherein the members are not affixed to the packages of prescription medication.

In accordance with another embodiment of the invention, a method of providing a plurality of prescription medication packages, each of which contains a medication, and approved Food and Drug Administration prescribing information specific for the prescription medication, the method comprises the steps of providing, in a container, a quantity of the prescription medication packages and providing, in the container, separately from the prescription medication packages, a quantity of members, each member comprising the approved Food and Drug Administration prescribing information specific for the prescription medication, the members being neither attached to nor contained within the prescription medication packages, wherein the quantity of members is equal to, or greater than, the quantity of the prescription medication packages.

In accordance with yet another embodiment of the invention, a method of preparing a container having a quantity of packages of prescription medication and approved Food and Drug Administration prescribing information specific for the packages of prescription medication, the method comprises the steps of providing the container, introducing the quantity of packages of prescription medication into the container, and introducing a quantity of members, each member comprising the approved Food and Drug Administration prescribing information specific for the packages of prescription medication into the container, wherein the quantity of the members is equal to, or greater than, the quantity of packages of prescription medication, and wherein the members are not affixed to the packages of prescription medication.

In accordance with another embodiment of the invention, a method of preparing a container having a plurality of prescription medication packages, each of which contains a medication, and approved Food and Drug Administration prescribing information specific for the prescription medication, the method comprises the steps of providing the container, introducing a quantity of the prescription medication packages into the container, and introducing, into the container, a quantity of members, each member comprising the approved Food and Drug Administration prescribing information specific for the prescription medication, the members being neither attached to nor contained within the prescription medication packages, wherein the quantity of members is equal to, or greater than, the quantity of the prescription medication packages.

In preferred embodiments, the package may be a bottle or a bottle in an individual box. The container may be a shipping carton. At least one member of the quantity of members may be printed onto a sheet of paper. In some embodiments, the sheet of paper is not folded. In other embodiments, the sheet of paper is folded at least once. The approved Food and Drug administration prescribing information may be presented as a machine readable code, for example, a QR code.

### BRIEF DESCRIPTION OF THE DRAWINGS

The foregoing features of embodiments will be more readily understood by reference to the following detailed description, taken with reference to the accompanying drawings, in which:

FIG. 1 shows an example of the prior art in which a folded sheet of paper containing FDA approved prescribing information is affixed to the top of a prescription medication package, here, a bottle.

FIG. 2 shows an exemplary illustration of a container having a quantity of prescription medication packages and an equal quantity of paper sheets containing the FDA approved prescribing information for the prescription medication in accordance with an embodiment of the present invention. The paper sheets containing the FDA approved prescribing information are not affixed to the prescription medication packages.

### DETAILED DESCRIPTION OF SPECIFIC EMBODIMENTS

Definitions. As used in this description and the accompanying claims, the following terms shall have the meanings indicated, unless the context otherwise requires.

As used herein, “approved Food and Drug Administration prescribing information,” “approved FDA prescribing information,” “Food and Drug Administration approved prescribing information,” “FDA approved prescribing information,” “approved prescribing information,” and the like, shall refer to the product label mandated by the Food and Drug Administration of the United States to be included with each package of prescription medication that is distributed by the medication's manufacturer. The approved prescribing information provides professional information (indications and usage, contraindications, warnings, and drug interactions, for example) about a medication to medical professionals, such as doctors and pharmacists. The approved prescribing information is typically presented on a single sheet of paper.

As used herein, “container” shall refer to any structure that is capable of containing and/or confining a plurality of prescription medication packages. Non-limiting examples of containers include cartons, shipping cartons, cases, shipping cases, boxes, crates, cans, and the like.

As used herein, “package of prescription medication,” “prescription medication package,” “package of medication,” “medication package,” and the like, shall refer to a structure that contains a commercial unit of a given prescription medication. Non-limiting examples of medication packages include boxes, bottles, a bottle in an individual box, and the like.

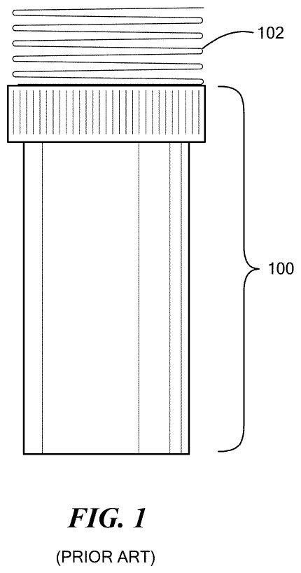

FIG. 1 shows an example of the prior art in which a folded sheet of paper comprising FDA approved prescribing information 102 is affixed to the top of prescription medication package 100. Here, prescription medication package 100 is a bottle, although it may also be a box or a bottle in an individual box, for example. The folded sheet of paper containing FDA approved prescribing information 102 may be affixed to prescription medication package 100 using an adhesive, e.g., glue.

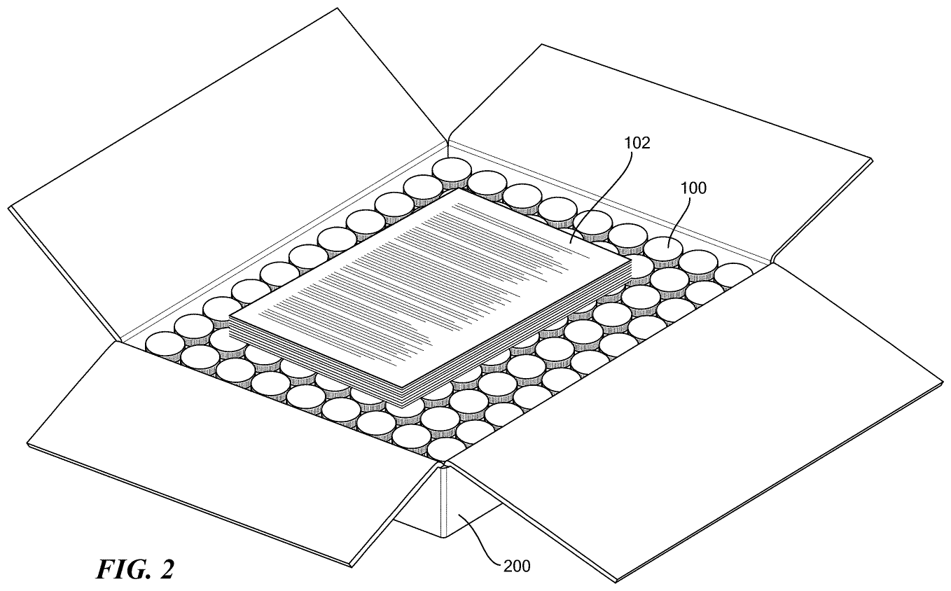

In accordance with an embodiment of the invention, FIG. 2 shows an exemplary illustration of a container 200 having a quantity of prescription medication packages 100 and an equal quantity of paper sheets comprising FDA approved prescribing information 102 specific for the prescription medication. The paper sheets comprising the FDA approved prescribing information 102 are laid atop of prescription medication packages 100.

In some embodiments, a container is provided that contains a quantity of prescription medication packages and a quantity of members comprising FDA approved prescribing information, specific for the prescription medication or the prescription medication packages, that is equal to, or greater than, the quantity of prescription medication packages, wherein the members are not affixed to, attached to, adhered to, or contained within the prescription medication packages.

In some embodiments, a method of providing a quantity of packages of prescription medication and approved Food and Drug Administration prescribing information specific for the packages of prescription medication, is provided. The method comprises the steps of providing, in a container, the quantity of packages of prescription medication and providing, in the container, a quantity of members, each member comprising the approved Food and Drug Administration prescribing information specific for the packages of prescription medication, wherein the quantity of the members is equal to, or greater than, the quantity of packages of prescription medication, and wherein the members are not affixed to the packages of prescription medication.

In some embodiments, a method of providing a plurality of prescription medication packages, each of which contains a medication, and approved Food and Drug Administration prescribing information specific for the prescription medication, is provided. The method comprises the steps of providing, in a container, a quantity of the prescription medication packages and providing, in the container, separately from the prescription medication packages, a quantity of members, each member comprising the approved Food and Drug Administration prescribing information specific for the prescription medication, the members being neither attached to nor contained within the prescription medication packages, wherein the quantity of members is equal to, or greater than, the quantity of the prescription medication packages.

In other embodiments, a method of preparing a container having a quantity of packages of prescription medication and approved Food and Drug Administration prescribing information specific for the packages of prescription medication, is provided. The method comprises the steps of providing the container, introducing the quantity of packages of prescription medication into the container, and introducing a quantity of members, each member comprising the approved Food and Drug Administration prescribing information specific for the packages of prescription medication into the container, wherein the quantity of the members is equal to, or greater than, the quantity of packages of prescription medication, and wherein the members are not affixed to the packages of prescription medication.

In some embodiments, a method of preparing a container having a plurality of prescription medication packages, each of which contains a medication, and approved Food and Drug Administration prescribing information specific for the prescription medication is provided. The method comprises the steps of providing the container, introducing a quantity of the prescription medication packages into the container, and introducing, into the container, a quantity of members, each member comprising the approved Food and Drug Administration prescribing information specific for the prescription medication, the members being neither attached to nor contained within the prescription medication packages, wherein the quantity of members is equal to, or greater than, the quantity of the prescription medication packages.

In some embodiments, the member is a sheet of paper. The FDA approved prescribing information may printed onto the sheet of paper.

In some embodiments, the container may be a carton, shipping carton, case, shipping case, box, crate, or can.

In some embodiments, the prescription medication package may be a box, bottle, or a bottle in an individual box.

In some embodiments, one or more members comprising the FDA approved prescribing information may be included within a container containing a quantity of prescription medication packages by laying a quantity of the members comprising the FDA approved prescribing information on top of the prescription medication packages. In some embodiments, the quantity of members comprising the FDA approved prescribing information may be laid beneath the prescription medication packages.

A computer and printer arrangement may be used to keep track the prescription medications being shipped in each container 200, and have all the required sheets containing approved prescribing information 102 automatically printed out. Then, the correct number of the required sheets may be placed in each container 200, even though different containers 200 may shipped with different numbers of and different types of prescription medication packages 100.

In some embodiments, the sheet or sheets of paper comprising the FDA approved prescribing information may be folded at least once. In other embodiments, the sheet or sheets of paper comprising the approved FDA approved prescribing information are not folded.

The prior art process of folding up each sheet of paper comprising the approved prescribing information 102 and then affixing each folded sheet to each prescription medication package 100 is an expensive process, in particular the affixing step. The present invention avoids these steps and thus provides significant savings in the cost of packaging and shipping prescription medications along with their FDA approved prescribing information.

In some embodiments, all or part of the FDA approved prescribing information may be presented on a sheet of paper as a machine readable code, for example a QR code. Upon scanning of the machine readable code with a smartphone or other device capable of reading such a code, the user is taken to a web page that provides all or part of the FDA approved prescribing information for a given prescription medication.

Various embodiments of the present invention may be characterized by the potential claims listed in the paragraphs following this paragraph (and before the actual claims provided at the end of this application). These potential claims form a part of the written description of this application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public.

Without limitation, potential subject matter that may be claimed (prefaced with the letter “P” so as to avoid confusion with the actual claims presented below) includes:

P1. A method of providing a package of prescription medication and its approved Food and Drug Administration prescribing information specific for the package of medication comprising:
providing, in a container, a quantity of the package of medication; and
providing, in the container, a quantity of the approved Food and Drug Administration prescribing information specific for the package of medication,
wherein the quantity of the approved Food and Drug Administration prescribing information is equal to, or greater than, the quantity of the package of medication, and
wherein the approved Food and Drug Administration prescribing information is not affixed to the package of medication.

P2. A method of providing a plurality of prescription medication packages, each of which contains a medication, and approved Food and Drug Administration prescribing information specific for the prescription medication, the method comprising:
providing, in a container, a quantity of the prescription medication packages; and
providing, in the container, separately from the prescription medication packages, a quantity of the approved Food and Drug Administration prescribing information specific for the prescription medication, the approved Food and Drug Administration prescribing information being neither attached to nor contained within the prescription medication packages,
wherein the quantity of the approved Food and Drug Administration prescribing information is equal to, or greater than, the quantity of the prescription medication packages.

P3. A method according to one of claim P1 or P2, wherein the package is a bottle or a bottle in an individual box.

P4. A method according to any of claims P1, P2 or P3, wherein the container is a shipping carton.

P5. A method according to any of claims P1-P4, wherein the approved Food and Drug Administration prescribing information is printed onto a single sheet of paper.

P6. A method according to claim P5, wherein the single sheet of paper is not folded.

P7. A method according to claim P5, wherein the single sheet of paper is folded at least once.

P8. A method according to any of claims P1-P7, wherein all or part of the approved Food and Drug administration prescribing information is presented as a machine readable code.

P9. A method according to claim P8, wherein the machine readable code is a QR code.

P10. A method of providing a quantity of packages of prescription medication and approved Food and Drug Administration prescribing information specific for the packages of prescription medication, the method comprising:
providing, in a container, the quantity of packages of prescription medication; and
providing, in the container, a quantity of members, each member comprising the approved Food and Drug Administration prescribing information specific for the packages of prescription medication,
wherein the quantity of members is equal to, or greater than, the quantity of packages of prescription medication, and
wherein the members are not affixed to the packages of prescription medication.

P11. A method of providing a plurality of prescription medication packages, each of which contains a medication, and approved Food and Drug Administration prescribing information specific for the prescription medication, the method comprising:
providing, in a container, a quantity of the prescription medication packages; and
providing, in the container, separately from the prescription medication packages, a quantity of members, each member comprising the approved Food and Drug Administration prescribing information specific for the prescription medication, the members being neither attached to nor contained within the prescription medication packages,
wherein the quantity of members is equal to, or greater than, the quantity of the prescription medication packages.

P12. A method of preparing a container having a quantity of packages of prescription medication and approved Food and Drug Administration prescribing information specific for the packages of prescription medication, the method comprising:
providing the container,
introducing the quantity of packages of prescription medication into the container; and
introducing a quantity of members, each member comprising the approved Food and Drug Administration prescribing information specific for the packages of prescription medication into the container,
wherein the quantity of members is equal to, or greater than, the quantity of packages of prescription medication, and
wherein the members are not affixed to the packages of prescription medication.

P13. A method of preparing a container having a plurality of prescription medication packages, each of which contains a medication, and approved Food and Drug Administration prescribing information specific for the prescription medication, the method comprising:
providing, the container,
introducing a quantity of the prescription medication packages into the container; and
introducing, into the container, a quantity of members, each member comprising the approved Food and Drug Administration prescribing information specific for the prescription medication, the members being neither attached to nor contained within the prescription medication packages,

wherein the quantity of members is equal to, or greater than, the quantity of the prescription medication packages.

P14. A method according to any one of claims P10-P13, wherein the package is a bottle or a bottle in an individual box.

P15. A method according to any one of claims P10-P14, wherein the container is a shipping carton.

P16. A method according to any of claims P10-P15, wherein at least one member of the quantity of members is a sheet of paper.

P17. A method according to claim P16, wherein the sheet of paper is not folded.

P18. A method according to claim P16, wherein the sheet of paper is folded at least once.

P19. A method according to any of claims P10-P18, wherein all or part of the approved Food and Drug administration prescribing information is presented as a machine readable code.

P20. A method according to claim P19, wherein the machine readable code is a QR code.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A method of providing a plurality of prescription medication packages, each of which contains a medication, and approved Food and Drug Administration prescribing information specific for the prescription medication, the method comprising:
providing, in a container, a quantity of the prescription medication packages; and
providing, in the container, separately from the prescription medication packages, a quantity of members, each member comprising the approved Food and Drug Administration prescribing information specific for the prescription medication, the members being neither attached to nor contained within the prescription medication packages,
wherein the quantity of members is equal to, or greater than, the quantity of the prescription medication packages.

2. A method according to claim 1, wherein the package is a bottle or a bottle in an individual box.

3. A method according to claim 1, wherein the container is a shipping carton.

4. A method according to claim 1, wherein at least one member of the quantity of members is a sheet of paper.

5. A method according to claim 4, wherein the sheet of paper is not folded.

6. A method according to claim 1, wherein all or part of the approved Food and Drug administration prescribing information is presented as a machine readable code.

7. A method according to claim 6, wherein the machine readable code is a QR code.

8. A method of preparing a container having a quantity of packages of prescription medication and approved Food and Drug Administration prescribing information specific for the packages of prescription medication, the method comprising:
providing the container,
introducing the quantity of packages of prescription medication into the container; and
introducing a quantity of members, each member comprising the approved Food and Drug Administration prescribing information specific for the packages of prescription medication into the container,
wherein the quantity of members is equal to, or greater than, the quantity of packages of prescription medication, and
wherein the members are not affixed to the packages of prescription medication.

9. A method according to claim 8, wherein the package is a bottle or a bottle in an individual box.

10. A method according to claim 8, wherein the container is a shipping carton.

11. A method according to claim 8, wherein at least one member of the quantity of members is a sheet of paper.

12. A method according to claim 8, wherein all or part of the approved Food and Drug administration prescribing information is presented as a machine readable code.

13. A method according to claim 12, wherein the machine readable code is a QR code.

14. A method of preparing a container having a plurality of prescription medication packages, each of which contains a medication, and approved Food and Drug Administration prescribing information specific for the prescription medication, the method comprising:
providing, the container,
introducing a quantity of the prescription medication packages into the container; and
introducing, into the container, a quantity of members, each member comprising the approved Food and Drug Administration prescribing information specific for the prescription medication, the members being neither attached to nor contained within the prescription medication packages,
wherein the quantity of members is equal to, or greater than, the quantity of the prescription medication packages.

15. A method according to claim 14, wherein the package is a bottle or a bottle in an individual box.

16. A method according to claim 14, wherein the container is a shipping carton.

17. A method according to claim 14, wherein at least one member of the quantity of members is a sheet of paper.

18. A method according to claim 17, wherein the sheet of paper is not folded.

19. A method according to claim 14, wherein all or part of the approved Food and Drug administration prescribing information is presented as a machine readable code.

20. A method according to claim 19, wherein the machine readable code is a QR code.
